(19) United States
(12) Patent Application Publication
(10) Pub. No.: US 2021/0007666 A1
(43) Pub. Date: Jan. 14, 2021

(54) BODY WATER CONTENT MEASUREMENT DEVICE, METHOD, AND PROGRAM

(71) Applicant: TERUMO KABUSHIKI KAISHA, Tokyo (JP)

(72) Inventors: Shinichiro SUDA, Hadano Kanagawa (JP); Kei HONDA, Hadano Kanagawa (JP)

(21) Appl. No.: 17/037,010

(22) Filed: Sep. 29, 2020

Related U.S. Application Data
(63) Continuation of application No. PCT/JP2019/011781, filed on Mar. 20, 2019.

(30) Foreign Application Priority Data
Mar. 29, 2018 (JP) 2018-064753

(51) Int. Cl. A61B 5/00; A61B 5/053

(52) U.S. Cl. CPC A61B 5/4878; A61B 5/7278; A61B 5/0537

(57) ABSTRACT

A body water content measurement device includes: a measurement section configured to generate a plurality of measurements corresponding to body water content of a subject over time; and an estimation section configured to estimate the body water content in a convergence state based on a plurality of body water content calculated respectively from the plurality of measurements.

### CROSS-REFERENCE TO RELATED APPLICATION(S)

This application is a continuation of International Patent Application No. PCT/JP2019/011781, filed on Mar. 20, 2019, which is based upon and claims the benefit of priority from Japanese Patent Application No. 2018-064753, filed on Mar. 29, 2018, the entire contents of which are incorporated herein by reference.

### BACKGROUND

### Technical Field

The present invention relates to a body water content measurement device, method, and program.

### Related Art

Measuring body water content is important for medical treatment or diagnosis of heart failure, renal failure, and the like, which cause congestion in the body. For example, JP 2005-131434 A discloses a device configured to measure the body water content using a bioimpedance method.

When a subject changes his or her body position during the measurement of body water content, water in the body moves along with the change of the body position, so that body water content cannot be measured accurately. As an example, when measuring the body water content using a bioimpedance method, water in the body moves so as to change the distribution of the water in the body, so that electrical resistance in the path through which the measurement current flows also changes. For this reason, there is a possibility that the body water content measured will be different from the actual body water content of the subject.

In the device disclosed in JP 2005-131434 A, there is no way to know whether or not the movement of the water in the body has subsided. Therefore, it is necessary to measure the body water content after a predetermined amount of time has passed from the time when the body position of the subject is kept constant. However, there are differences in the convergence time during which the movement of the water in the body subsides for different individuals. Therefore, if the waiting time from the time when the body position of the subject is kept constant, is set uniformly, there is a possibility that the waiting time is insufficient depending on the individual and in such cases the body water content cannot be measured accurately.

### SUMMARY

In view of the aforementioned circumstances, embodiments provide a measurement device, a measurement method, and a measurement program for measuring the body water content more accurately.

A body water content measurement device according to an embodiment includes: a measurement section configured to generate a plurality of measurements corresponding to body water content of a subject over time; and an estimation section configured to estimate the body water content in a convergence state based on a plurality of body water content calculated respectively from the plurality of measurements.

A body water content measurement method according to an embodiment includes: generating a plurality of measurement corresponding to body water content of a subject over time; and estimating the body water content in a convergence state based on a plurality of body water content calculated respectively from the plurality of measurements.

A body water content measurement program according to an embodiment is executable on a processor to carry out a procedure for acquiring a plurality of measurements corresponding to body water content of a subject over time; and a procedure for estimating the body water content in a convergence state based on a plurality of body water content calculated respectively from the plurality of measurements.

According to embodiments, since the body water content in the convergence state is estimated using the values of the time-dependent body water content, it is possible to determine the body water content more accurately compared with a case where the body water content is determined by setting a uniform waiting time after the body position of the subject is kept constant.

### BRIEF DESCRIPTION OF DRAWINGS

FIG. 1 is a schematic diagram of a measurement device according to a first embodiment;

FIG. 2 is a block diagram of the measurement device according to the first embodiment;

FIG. 3 is a diagram describing a method of estimating the body water content by the measurement device according to the first embodiment;

FIG. 4 is a flowchart illustrating a measurement method according to the first embodiment;

FIG. 5 is a flowchart illustrating a measurement method according to a second embodiment;

FIG. 6 is a diagram describing a method of estimating the body water content in the measurement method according to the second embodiment; and

FIG. 7 is a flowchart illustrating a measurement method according to a modification example of the second embodiment.

### DETAILED DESCRIPTION

Hereinafter, embodiments will be described with reference to the accompanying drawings. In addition, in the description of the drawings, the same elements are denoted by the same reference numerals, and the repeated description thereof will be omitted. In addition, the dimensional ratios in the drawings are exaggerated for convenience of explanation, and may be different from the actual ratios.

### First Embodiment

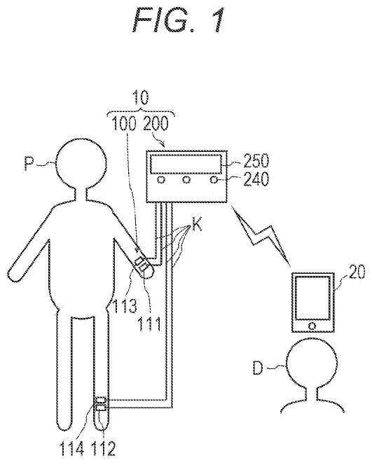

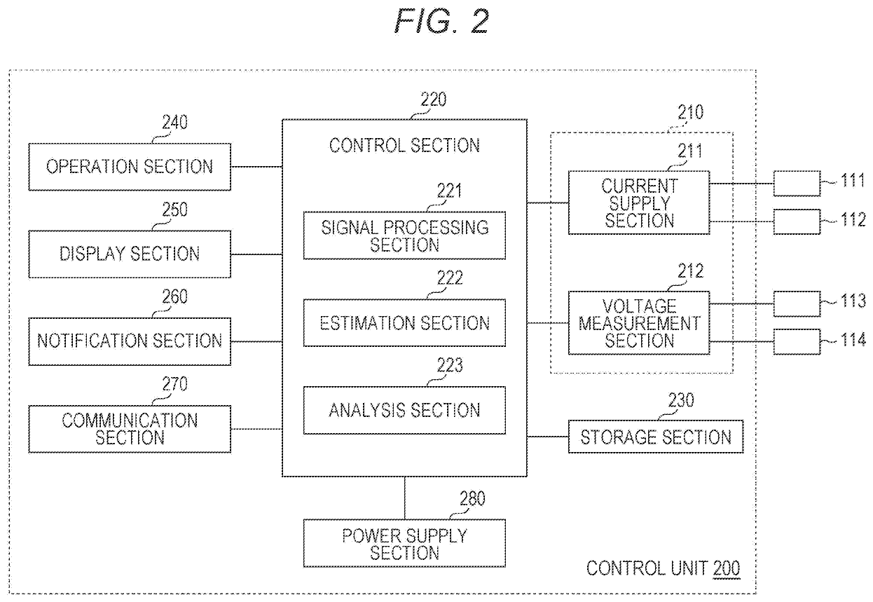

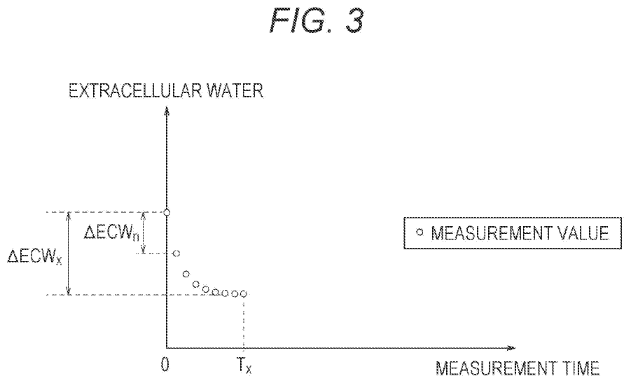

FIGS. 1 and 2 are diagrams describing the configuration of a measurement device 10 according to a first embodiment. FIG. 3 is a diagram describing a method of estimating the body water content by the measurement device 10 according to the first embodiment.

The measurement device 10 according to the first embodiment is a device that measures the body water content of a subject P who is, for example, a patient with heart failure or renal failure. As used herein, the body water content may be, for example, extracellular water (ECW), intracellular water (ICW), or the total amount of body water that is the sum of the extracellular water and the intracellular water.

The measurement device 10 is particularly useful during the treatment stage of heart failure after the acute phase. A treatment for heart failure after the acute phase includes treatments with diuretics or the like to remove excess body water accumulated in the body in order to reduce the burden on the heart or kidneys. A medical staff, such as a doctor, can prescribe diuretics appropriately based on the body water content measured by the measurement device 10. As a result, excess body water in the patient's body can be removed more efficiently.

The measurement device 10 according to the first embodiment will be briefly described with reference to FIG. 1. The measurement device 10 includes an electrode unit 100 and a control unit 200 connected to the electrode unit 100 through a cable K. Hereinafter, each section of the measurement device 10 will be described.

(Electrode Unit)

The electrode unit 100 includes a pair of current application electrodes 111 and 112 attached to the body of the subject P by which a current is passed through the body of the subject P and a pair of measurement electrodes 113 and 114 attached to the body of the subject P by which the voltage of the body of the subject P is measured. Hereinafter, each section of the electrode unit 100 will be described in detail.

The current application electrode 111 is attached to the wrist of the subject P in the present embodiment. The current application electrode 112 is attached to the ankle of the subject P in the present embodiment. However, the attachment positions of the pair of current application electrodes 111 and 112 can be appropriately selected according to a part (whole body, back, arm, leg, or the like) for which bioimpedance needs to be measured.

As illustrated in FIG. 2, the pair of current application electrodes 111 and 112 are electrically connected to a current supply section 211 of a measurement section 210 described later. The current application electrodes 111 and 112 are used to pass an AC current from the wrist of the subject P in contact with the current application electrode 111 to the ankle of the subject P in contact with the current application electrode 112 (or in the opposite direction).

As illustrated in FIG. 1, the measurement electrode 113 is attached to the wrist of the subject P in the present embodiment. The measurement electrode 114 is attached to the ankle of the subject P in the present embodiment. However, the attachment positions of the pair of measurement electrodes 113 and 114 can be appropriately selected according to a part (whole body, back, arm, leg, or the like) for which bioimpedance needs to be measured.

As illustrated in FIG. 2, the measurement electrodes 113 and 114 are electrically connected to a voltage measurement section 212 of the measurement section 210 described later. The measurement electrodes 113 and 114 are used to measure the voltage difference between the wrist of the subject P in contact with the measurement electrode 113 and the ankle of the subject P in contact with the measurement electrode 114 when the AC current is supplied to the pair of current application electrodes 111 and 112.

(Control Unit)

As illustrated in FIG. 2, the control unit 200 includes the measurement section 210, a control section 220, a storage section 230, an operation section 240, a display section 250, a notification section 260, a communication section 270, and a power supply section 280. Hereinafter, each section of the control unit 200 will be described in detail.

First, the measurement section 210 will be described.

The measurement section 210 is for example a measurement circuit and includes the current supply section 211 and the voltage measurement section 212. The method employed by the measurement section 210 for voltage measurement includes the Tetra-polar method, and the cole-cole plot method.

The current supply section 211 supplies an AC current to the body of the subject P through the current application electrodes 111 and 112. The current supply section 211 includes a known AC power source that generates an AC current or the like.

The voltage measurement section 212 measures a voltage difference between the measurement electrodes 113 and 114 when the AC current is supplied to the pair of current application electrodes 111 and 112. The voltage measurement section 212 may be any known voltage measurement device. The supply of the AC current by the current supply section 211 and the measurement of the voltage by the voltage measurement section 212 are performed at predetermined time intervals while keeping the body position of the subject P constant.

Next, the control section 220 will be described.

The control section 220 is for example a controller circuit and includes a processor, such as a central processing unit (CPU). The control section 220 is electrically connected to the measurement section 210, the storage section 230, the operation section 240, the display section 250, the notification section 260, the communication section 270, the power supply section 280, and the like, and controls their operations.

The control section 220 executes a measurement program stored in the storage section 230 to function as a signal processing section 221, an estimation section 222, and an analysis section 223 (e.g., “time analysis section” and/or “body water content analysis section”).

The signal processing section 221 calculates the bioimpedance of the subject P based on a current value of the AC current supplied by the current supply section 211 and a voltage difference measured by the voltage measurement section 212. In addition, in the present embodiment, the signal processing section 221 calculates the extracellular water from the calculated bioimpedance, and the height, weight, sex, age, and the like of the subject P, which are input through the operation section 240. In addition, since a specific method for calculating the bioimpedance and the extracellular water is known, the description thereof will be omitted. The calculated extracellular water is stored in the storage section 230.

The estimation section 222 estimates the extracellular water in a convergence state, based on the measured time-dependent extracellular water. As used herein, in the “convergence state” means a state in which: (1) a sufficient amount of time has passed since the body position of the subject P has been kept constant, (2) movement of body water in the body of the subject P has subsided, and (3) a time variation in the body water content (extracellular water in the present embodiment) measured in the body of the subject P falls within the range of the measurement error.

The extracellular fluid contains blood, lymph fluid, interstitial fluid, and the like, and is partitioned over a wider region than the intracellular fluid partitioned by the cell membrane. For this reason, the extracellular fluid is more likely to move due to the influence of gravity when the subject P changes his or her body position than the intracellular fluid. In addition, in heart failure or renal failure, the extracellular water stored in the body of the subject P tends to increase significantly. Therefore, the extracellular fluid is more likely to move as the body position of the subject P changes. When measuring the extracellular water using a bioimpedance method, the extracellular fluid moves in the body of the subject P, and the distribution of the extracellular water in the body changes, so that the electrical resistance in the path through which the measurement current flows, changes. Therefore, the measured value of the extracellular water in the subject may be different from the actual extracellular water in the subject. For this reason, estimating the extracellular water in the convergence state by the measurement device 10 is particularly useful in measuring the extracellular water during the treatment of heart failure or renal failure.

In addition, when the treatment of heart failure or renal failure progresses and the excess extracellular water stored in the body decreases, the patient performs rehabilitation. Therefore, after the patient performs rehabilitation such as walking, the extracellular water may be measured after moving the patient to a body position such as a recumbent position. In such measurement after rehabilitation, the extracellular fluid in the patient's body is likely to move. For this reason, estimating the extracellular water in the convergence state by the measurement device 10 is also useful in measuring the extracellular water after rehabilitation. It should be noted that the measurement timing of the measurement device 10 is not limited to after rehabilitation.

As shown in FIG. 3, the estimation section 222 calculates a difference ΔECWₙ between the extracellular water measured at the n-th time and the extracellular water measured at the (n−1)-th time, where n≥2. In addition, in FIG. 3, as an example, the difference ΔECWₙ between the extracellular water measured at the first time and the extracellular water measured at the second time is shown.

When the difference ΔECWₙ between the extracellular water measured at the n-th time and the extracellular water measured at the (n−1)-th time is equal to or less than the reference value, the estimation section 222 estimates the extracellular water measured at the n-th time as the extracellular water in the convergence state. When the difference ΔECWₙ is larger than the reference value, the measurement of the extracellular water by the measurement section 210, the calculation of the difference ΔECWₙ by the estimation section 222, and the determination as to whether or not the calculated difference ΔECWₙ is equal to or less than the reference value are repeated until the difference ΔECWₙ becomes equal to or less than the reference value. The reference value is not limited to a particular value and may be any value so long as the estimation section 222 can determine that the convergence state has been reached based on such value. For example, the reference value can be set to a value of 0.1 kg or less.

The estimation section 222 may set the difference ΔECWₙ that is equal to or less than the reference value as the estimation accuracy.

The analysis section 223 estimates a convergence time Tₓ shown in FIG. 3 as the time from the start of measurement by the measurement section 210 to the onset of the convergence state. More particularly, the analysis section 223 estimates, as the convergence time Tₓ, a time from the measurement of the extracellular water measured at the first time to the measurement of the extracellular water at which the difference ΔECWₙ becomes equal to or less than the reference value.

The analysis section 223 estimates the amount of change ΔECWₓ in the extracellular water from the start of measurement of the extracellular water by the measurement section 210 to the onset of the convergence state. In the present embodiment, the analysis section 223 estimates, as the amount of change ΔECWₓ in the extracellular water until the convergence state is reached, the difference between the measurement value of the extracellular water measured at the first time and the value of the extracellular water estimated as the extracellular water in the convergence state.

Next, the storage section 230 will be described.

The storage section 230 includes a read only memory (ROM) that stores various programs or various kinds of data, a random access memory (RAM) that temporarily stores programs or data, a hard disk that stores various programs including an operating system or various kinds of data. The storage section 230 stores a measurement program for estimating the extracellular water in the convergence state and various kinds of data used in the execution of the measurement program. The measurement program may be provided by a computer-readable recording medium in which the measurement program is stored, or may be downloaded from the Internet. The recording medium is not limited to any particular type so long as the recording medium can be read by a computer. For example, the recording medium can be an optical disk such as a CD-ROM or a DVD-ROM, a USB memory, an SD memory card, and the like.

Next, the operation section 240 will be described.

In the present embodiment, as illustrated in FIG. 1, the operation section 240 is includes a plurality of operation buttons. By operating the operation section 240, the user can input information regarding the subject P, such as height, weight, sex, and age, instruct the measurement device 10 to start measurement, and set a time interval (sampling cycle) for the measurement. By operating the operation section 240, the user can perform other settings for measuring the extracellular water.

Next, the display section 250 will be described.

In the present embodiment, as illustrated in FIG. 1, the display section 250 includes a liquid crystal display. The display section 250 displays the value estimated by the estimation section 222 as the extracellular water in the convergence state. The display section 250 may display the value that is set as the estimation accuracy by the estimation section 222 (e.g., ΔECWₙ equal to or less than the reference value). The display section 250 displays the convergence time Tₓ calculated by the analysis section 223 and the amount of change ΔECWₓ in the extracellular water until the convergence state is reached. The display section 250 may display a graph plotting the measured time-dependent extracellular water as shown in FIG. 3. The display section 250 displays other pieces of information provided for measuring the extracellular water.

In addition, the configurations of the operation section 240 and the display section 250 are not limited to the above. For example, the operation section 240 and the display section 250 may be integrally configured as a touch panel.

Next, the notification section 260 will be described.

The notification section 260 is not particularly limited to any particular structure so long as it is able to provide a notification that the estimation of the extracellular water in the convergence state by the estimation section 222 has completed. For example, the notification section 260 may be a speaker or the like that sounds a buzzer when the estimation of the extracellular water in the convergence state by the estimation section 222 has completed. In addition, the display section 250 may function as a notification section by displaying that the convergence state has been reached. Alternatively, the notification section 260 may be a device external to the measurement device 10. For example, the notification section 260 may be an operation terminal 20 of a measurer D, that receives from the measurement device 10 a signal indicating that the estimation of the extracellular water in the convergence state by the estimation section 222 has completed and in turn provides the notification to the measurer D or the like.

Next, the communication section 270 will be described.

The communication section 270 is an interface circuit for wirelessly communicating with an external device. The external device is not limited to any particular type. For example, as illustrated in FIG. 1, the operation terminal 20 of the measurer D (for example, a medical staff such as a doctor or a nurse) can be the external device. The communication section 270 transmits, to the operation terminal 20 of the measurer D, the value estimated by the estimation section 222 as the extracellular water in the convergence state. In addition, the communication section 270 may transmit the value that is set as the estimation accuracy by the estimation section 222 (e.g., ΔECWₙ equal to or less than the reference value) or the convergence time Tₓ calculated by the analysis section 223 and the amount of change ΔECWₓ in the extracellular water until the convergence state is reached. Thus, the measurer D may check the measurement result of the measurement device 10 on the operation terminal 20 instead of the control unit 200. Alternatively, the measurement device 10 does not include the communication section 270, and the measurement result of the measurement device 10 is displayed only on the display section 250.

Next, the power supply section 280 will be described.

The power supply section 280 is not limited to any particular type. For example, the power supply section 280 may be a battery or a voltage converter that converts a voltage supplied from a commercial power supply into a predetermined voltage and supply the voltage to each section.

(Measurement Method)

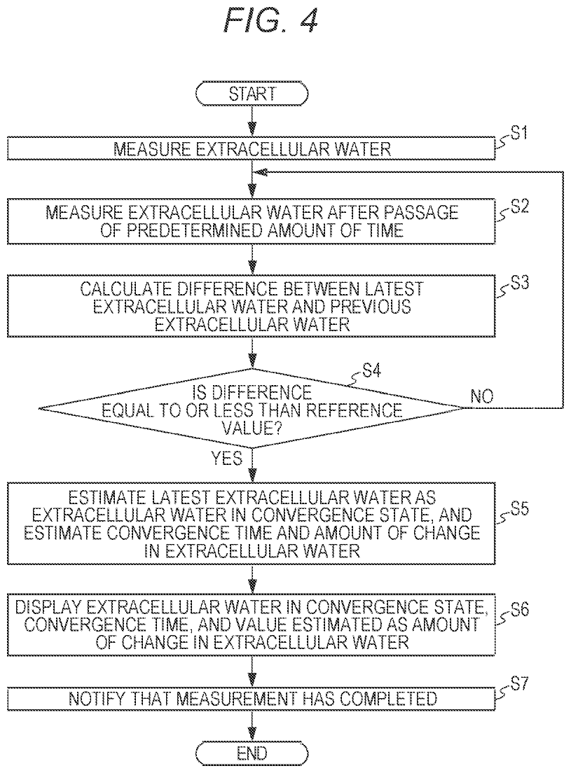

FIG. 4 is a flowchart describing a measurement method according to the first embodiment.

The measurement method according to the first embodiment will be briefly described with reference to FIG. 4. In the measurement method according to the first embodiment, while the extracellular water of the subject P is measured over time (steps S1 and S2), it is determined whether or not the movement of the extracellular fluid in the body of the subject P has subsided based on the measured time-dependent extracellular water (steps S3 and S4). When it is determined that the movement of the extracellular fluid has subsided, the latest measured extracellular water is estimated as the extracellular water in the convergence state and the convergence time Tₓ and the amount of change ΔECWₓ in the extracellular water until the convergence state is reached are estimated (step S5). In addition, the extracellular water in the convergence state, the convergence time Tₓ, and the value estimated as the amount of change ΔECWₓ in the extracellular water are displayed (step S6), and notification indicating that the measurement has completed is provided (step S7). Hereinafter, the measurement method will be described in detail.

Before the measurement by the measurement device 10 is started, first, the measurer D keeps the body position of the subject P constant. Then, the measurer D attaches the electrode unit 100 to the body of the subject P, as illustrated in FIG. 1. Then, the measurer D operates the operation section 240 to instruct the measurement device 10 to start measuring the extracellular water.

Therefore, the control section 220 causes the measurement section 210 to measure the extracellular water for the first time (step S1, refer to FIG. 4).

Then, the control section 220 causes the measurement section 210 to measure the extracellular water for the second time after the passage of a predetermined amount of time from the previous measurement (step S2).

Then, the estimation section 222 calculates the difference ΔECWₙ between the previously measured extracellular water and the latest measured extracellular water (step S3).

Then, the estimation section 222 determines whether or not the difference ΔECWₙ between the previously measured extracellular water and the latest measured extracellular water is equal to or less than the reference value (step S4).

When the difference ΔECWₙ is not equal to or less than the reference value (S4; No), the control section 220 repeatedly performs steps S2 to S4 until the difference ΔECWₙ becomes equal to or less than the reference value.

When it is determined that the difference ΔECWₙ is equal to or less than the reference value (S4; Yes), the estimation section 222 estimates the measurement value of the latest measured extracellular water as the extracellular water in the convergence state (step S5). At this time, the estimation section 222 may set the difference ΔECWₙ equal to or less than the reference value as the estimation accuracy. Then, the analysis section 223 estimates, as the convergence time Tₓ, a time from the first measurement of the extracellular water to the measurement of the extracellular water at which the difference ΔECWₙ becomes equal to or less than the reference value. In addition, the analysis section 223 estimates, as the amount of change ΔECWₓ in the extracellular water until the convergence state is reached, the difference between the measurement value of the extracellular water measured at the first time and the value of the extracellular water estimated as the extracellular water in the convergence state.

Then, the control section 220 displays, on the display section 250, the extracellular water in the convergence state, the estimation accuracy, the convergence time Tₓ, and the value estimated as the amount of change ΔECWₓ in the extracellular water until the convergence state is reached (step S6). In addition, at this time, the display section 250 may display a graph plotting the measured time-dependent extracellular water as shown in FIG. 3. In addition, at this time, the control section 220 may transmit the extracellular water in the convergence state, the estimation accuracy, the convergence time Tₓ, and the value estimated as the amount of change ΔECWₓ in the extracellular water until the convergence state is reached, to the operation terminal 20 of the measurer D through the communication section 270.

For example, the larger the extracellular water in the body due to differences in physique, gender, and the like, the larger the amount of movement of the extracellular fluid, so that it takes time for the movement of the extracellular fluid to subside. In addition, for example, the more the extracellular water is accumulated due to heart failure or renal failure, the larger the amount of movement of the extracellular fluid, so that it takes time for the movement of the extracellular fluid to subside. In addition, healthy persons or patients performing rehabilitation during hospitalization have a large amount of movement compared with bedridden patients. Therefore, since the amount of movement of the extracellular fluid in healthy persons or patients performing rehabilitation during hospitalization is large, it takes time for the extracellular fluid to subside. As described above, there are individual differences in the amount of time during which the movement of the extracellular fluid subsides. Therefore, the time required for the movement of the extracellular water to subside changes between subjects. For this reason, if the body water content is measured with a uniform waiting time from the time when the body position of the subject is kept constant, the waiting time may be longer than necessary depending on the condition of the subject, and would waste the subject's time. In addition, depending on the condition of the subject, there is a possibility that the required waiting time is too short and the accurate extracellular water cannot be measured accurately. The measurement device 10 according to the present embodiment determines whether or not the movement of body water in the body of the subject has subsided based on the difference ΔECWₙ between the n-th extracellular water and the (n−1)-th extracellular water. Therefore, it is possible to reduce unnecessary waiting time while ensuring a more accurate measurement of the extracellular water.

In addition, the measurement device 10 according to the present embodiment causes the display section 250 to display the convergence time Tₓ and the amount of change ΔECWₓ in the extracellular water until the convergence state is reached. For example, when the convergence time Tₓ is relatively long and/or the amount of change ΔECWₓ in the extracellular water is relatively large, this may be an indication that the condition of the subject P, who is a heart failure patient, has deteriorated. Therefore, the measurer D such as a doctor or a nurse can easily check the change in the condition of the subject P due to heart failure, renal failure, or the like, using the convergence time Tₓ and the amount of change ΔECWₓ in the extracellular water.

Then, the control section 220 causes the notification section 260 to notify that the estimation section 222 has completed the estimation of the extracellular water in the convergence state (measurement has completed) (step S7). Therefore, the subject P and/or the measurer D who is the user of the measurement device 10 can know that the measurement has completed. As a result, the subject P is released from the requirement to keep the body position constant.

In addition, steps S6 and S7 may be performed simultaneously. In addition, in step S4, in addition to the determination as to whether or not the difference ΔECWₙ is equal to or less than the reference value, it may be determined whether or not a predetermined amount of time has passed from the start of measurement of the extracellular water by the measurement section 210. Then, when a predetermined amount of time has passed from the start of measurement and the difference ΔECWₙ is equal to or less than the reference value, the measurement device 10 may perform the processing from step S5. Thus, when the difference ΔECWₙ accidentally becomes equal to or less than the reference value immediately after the start of measurement, it is possible to prevent the extracellular water, for which the difference ΔECWₙ accidentally becomes equal to or less than the reference value, from being adopted as the extracellular water in the convergence state. In addition, when the difference ΔECWₙ does not become equal to or less than the reference value even after a predetermined amount of time (for example, 20 minutes) has passed from the start of measurement, the estimation section 222 may stop the estimation operation, and the control section 220 may display the measurement value of the latest extracellular water as a reference value on the display section 250 of the control unit 200 or the display section of the operation terminal 20 of the measurer D together with the fact that the estimation operation has stopped.

As described above, the measurement device 10 according to the first embodiment includes the measurement section 210 that measures the body water content of the subject P over time and the estimation section 222 that estimates, based on the time-dependent body water content measured by the measurement section 210, the body water content in the convergence state in which the movement of body water in the body of the subject P has subsided.

According to the measurement device 10 described above, since the body water content in the convergence state is estimated using the measurement value of the time-dependent body water content, it is possible to measure the body water content more accurately compared with a case where a uniform waiting time from the time when the body position of the subject P is kept constant is required prior to performing the measurement.

In addition, the estimation section 222 calculates the difference ΔECWₙ between the n-th body water content and the (n−1)-th body water content of the time-dependent body water content. When the difference ΔECWₙ is equal to or less than the reference value, the estimation section 222 estimates the n-th body water content as the body water content in the convergence state. In this manner, the measurement device 10 can determine whether or not the movement of body water in the body of the subject P has subsided based on the difference ΔECWₙ.

In addition, the measurement device 10 further includes the analysis section 223 that estimates the convergence time Tₓ from the start of measurement by the measurement section 210 to the onset of the convergence state. Therefore, the measurer D can check the state of body water of the subject P easily and accurately using the convergence time Tₓ.

In addition, the measurement device 10 further includes the analysis section 223 that estimates the amount of change ΔECWₓ in the body water content until the convergence state is reached. Therefore, the measurer D can check the state of body water of the subject easily and accurately using the amount of change ΔECWₓ in the body water content.

In addition, the measurement device 10 further includes the notification section 260 that notifies that the estimation section 222 has completed the estimation of the body water content in the convergence state. Therefore, the subject P and the measurer D can know that the measurement has completed.

In addition, the measurement section 210 measures the bioimpedance of the subject P by supplying a current to a pair of current application electrodes 111 and 112 attached to the body of the subject P and measuring the voltage of a pair of measurement electrodes 113 and 114 attached to the body of the subject P. Therefore, the measurement section 210 can measure the body water content of the subject P using the bioimpedance method.

In addition, the body water content includes the extracellular water. Therefore, the measurement device 10 can more accurately measure the value of the extracellular water useful in the diagnosis of heart failure, renal failure, and the like.

In addition, in the measurement method according to the first embodiment, the body water content of the subject P is measured over time (steps S1 and S2), and the body water content in the convergence state in which the movement of body water in the body of the subject P has subsided is estimated based on the measured time-dependent body water content (steps S3 to S5).

In addition, the measurement program according to the first embodiment executes a procedure of measuring the body water content of the subject P over time and a procedure of estimating, based on the measured time-dependent body water content, the body water content in the convergence state in which the movement of body water in the body of the subject P has subsided.

According to the measurement method and the measurement program described above, the body water content in the convergence state is estimated using the measurement value of the time-dependent body water content. Therefore, it is possible to measure the body water content more accurately compared with a case where a uniform waiting time from the time when the body position of the subject P is kept constant is required prior to performing the measurement.

### Second Embodiment

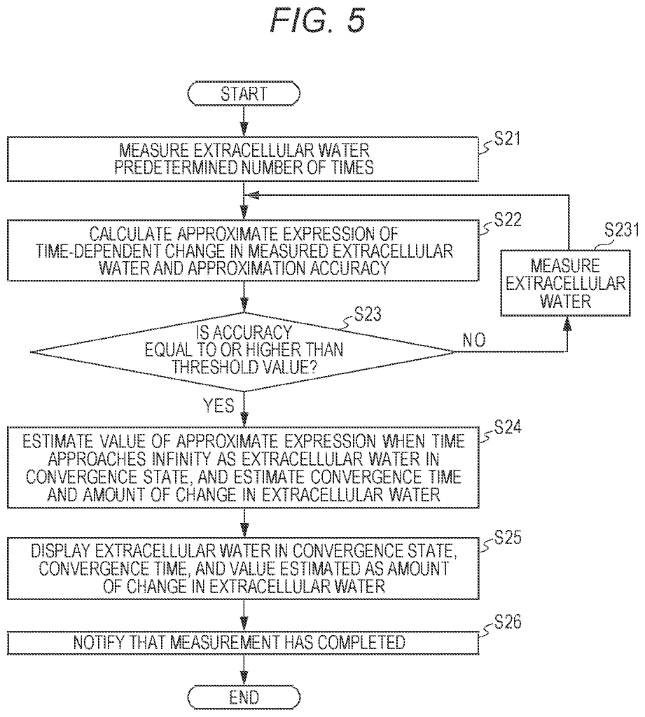

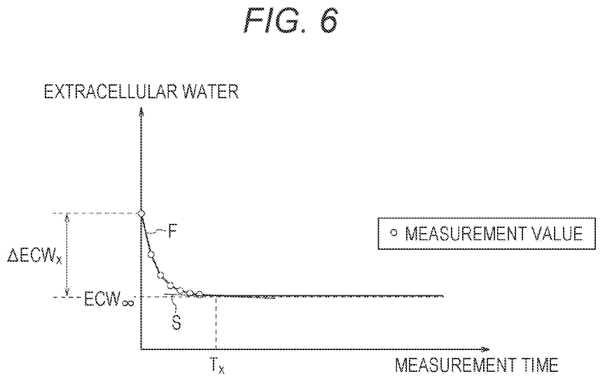

FIG. 5 is a flowchart illustrating a measurement method according to a second embodiment. FIG. 6 is a diagram describing a method of estimating the body water content in a convergence state in the measurement method according to the second embodiment.

The measurement device 10 and the measurement method according to the second embodiment are different from those according to the above embodiment in a method of estimating the body water content in the convergence state. Hereinafter, the measurement device 10 and the measurement method according to the second embodiment will be described. In addition, since the configuration of the measurement device 10 according to the second embodiment is the same as the configuration of the measurement device 10 according to the first embodiment except for the processing methods of the estimation section 222 and the analysis section 223, the description of the configuration will be omitted.

The measurement method according to the second embodiment will be briefly described with reference to FIG. 5. In the measurement method according to the second embodiment, the extracellular water of the subject P is measured a predetermined number of times (step S21), Approximate Expression F for approximating a time-dependent change in the measured extracellular water is calculated (step S22), a convergence value ECW_{∞}, which is the value of Approximate Expression F when time approaches infinity, is estimated as the extracellular water in the convergence state, and the convergence time Tₓ and the amount of change ΔECWₓ in the extracellular water until the convergence state is reached, are estimated (step S24), the extracellular water in the convergence state, the convergence time Tₓ, and the value estimated as the amount of change ΔECWₓ in the extracellular water until the convergence state is reached, are displayed (step S25), and notification indicating that the measurement has completed is provided (step S26). Hereinafter, the measurement method according to the second embodiment will be described in detail.

Before the measurement by the measurement device 10 is started, first, the measurer D keeps the position of the subject P constant. Then, the measurer D attaches the electrode unit 100 to the body of the subject P, as illustrated in FIG. 1. Then, a doctor, a nurse, or the like operates the operation section 240 to instruct the measurement device 10 to start measuring the extracellular water.

Therefore, the control section 220 causes the measurement section 210 to measure the extracellular water at predetermined time intervals for a predetermined amount of time (for example, about 3 to 5 minutes) (step S21, refer to FIG. 5). As a result, the control section 220 acquires the measurement value of the extracellular water a predetermined number of times.

Then, the estimation section 222 calculates Approximate Expression F for approximating the time-dependent change in the measured extracellular water and the approximation accuracy according to known methods, for example, the time series analysis method (step S22). According to the research of the inventors, it has been found out that the measurement value of the extracellular water converges to a constant value with the passage of a certain amount of time after the body position of the subject P is kept constant. Therefore, as shown in FIG. 6 as an example, Approximate Expression F can be set to an expression that converges to a constant value as time approaches infinity. In addition, although a case where the extracellular water gradually decreases from the start of the measurement and then converges to a constant value is shown as an example in FIG. 6, the extracellular water may gradually increase and then converge to a constant value.

The method of calculating Approximate Expression F is not limited to any particular method. For example, a known regression analysis method such as the least squares method can be used. In addition, the approximation accuracy is not limited to any particular type. For example, the approximation accuracy can be expressed by a determination coefficient. In addition, the estimation section 222 may calculate a plurality of types of Approximate Expressions F and select Approximate Expression F with the highest approximation accuracy among these.

Then, the estimation section 222 determines whether or not the approximation accuracy is equal to or higher than a threshold value (step S23). The threshold value is not limited to any particular value so long as the approximation accuracy can be guaranteed. For example, when the accuracy is expressed by a determination coefficient, the threshold value can be set to a value of 0.8 or more.

When the approximation accuracy is not equal to or higher than the threshold value (S23; No), the control section 220 causes the measurement section 210 to measure the (n+1)-th extracellular water (step S231). Then, the control section 220 calculates Approximate Expression F and the approximation accuracy of the measurement value of the extracellular water up to the (n+1)-th time (step S22), and determines whether or not the approximation accuracy is equal to or higher than the threshold value (step S23). The control section 220 repeats steps S231, S22, and S23 until the approximation accuracy becomes equal to or higher than the threshold value.

When the approximation accuracy is equal to or higher than the threshold value (S23; Yes), the estimation section 222 calculates the convergence value ECW_{∞}, which is the value of Approximate Expression F when time approaches infinity (refer to FIG. 6), and estimates the calculated convergence value ECW_{∞} as the extracellular water in the convergence state (step S24). In addition, the method of calculating the convergence value ECW_{∞} is not particularly limited. For example, a method of designating infinity as an input value of the time in Approximate Expression F and a method of inputting a finite value that is large enough to be treated as infinity in Approximate Expression F (for example, a maximum value that can be treated by the programming language of the measurement program) can be used.

Then, as shown in FIG. 6, the analysis section 223 estimates, as the convergence time Tₓ, the time when the slope of a tangent line S in Approximate Expression F reaches a predetermined value. The predetermined value is not particularly limited as long as this is a value close to 0 (zero) to the extent that the convergence state can be determined. In addition, the analysis section 223 estimates, as the amount of change in the extracellular water until reaching the convergence state, the difference between the convergence value ECW_{∞} and the extracellular water measured at the first time.

Then, the control section 220 displays, on the display section 250, the extracellular water in the convergence state, the approximation accuracy, the convergence time Tₓ, and the value estimated as the amount of change ΔECWₓ in the extracellular water (step S25). In addition, at this time, the display section 250 may display a graph plotting the measured time-dependent extracellular water and Approximate Expression F as shown in FIG. 6, a graph plotting the time change of the tangent line S in Approximate Expression F, and the like.

Then, the control section 220 causes the notification section 260 to notify that the estimation section 222 has completed the estimation (measurement has completed) (step S26).

In addition, steps S25 and S26 may be performed simultaneously. In addition, when the approximation accuracy does not become equal to or higher than the threshold value even if a predetermined amount of time (for example, 20 minutes) has elapsed from the start of measurement, the estimation section 222 may stop the estimation operation, and the control section 220 may display the measurement value of the latest extracellular water as a reference value on the display section 250 of the control unit 200 or the display section of the operation terminal 20 of the measurer D together with the fact that the estimation operation has stopped.

As described above, in the measurement device 10 according to the second embodiment, the estimation section 222 calculates Approximate Expression F for approximating the time-dependent body water content measured by the measurement section 210 and estimates, as the body water content in the convergence state, the convergence value ECW_{∞}, which is the value of Approximate Expression F when time approaches infinity. For this reason, the measurement device 10 according to the second embodiment can estimate the body water content in the convergence state even if the movement of body water in the body of the subject P has not subsided. Therefore, the measurement device 10 according to the second embodiment can complete the measurement in a shorter time as compared with the first embodiment.

In addition, the estimation section 222 calculates the approximation accuracy of Approximate Expression F. When the approximation accuracy is equal to or higher than the threshold value, the estimation section 222 sets, as the body water content in the convergence state, the convergence value ECW_{∞} when the time approximates infinity in Approximate Expression F. Therefore, the reliability of approximation can be ensured.

### Modification Examples

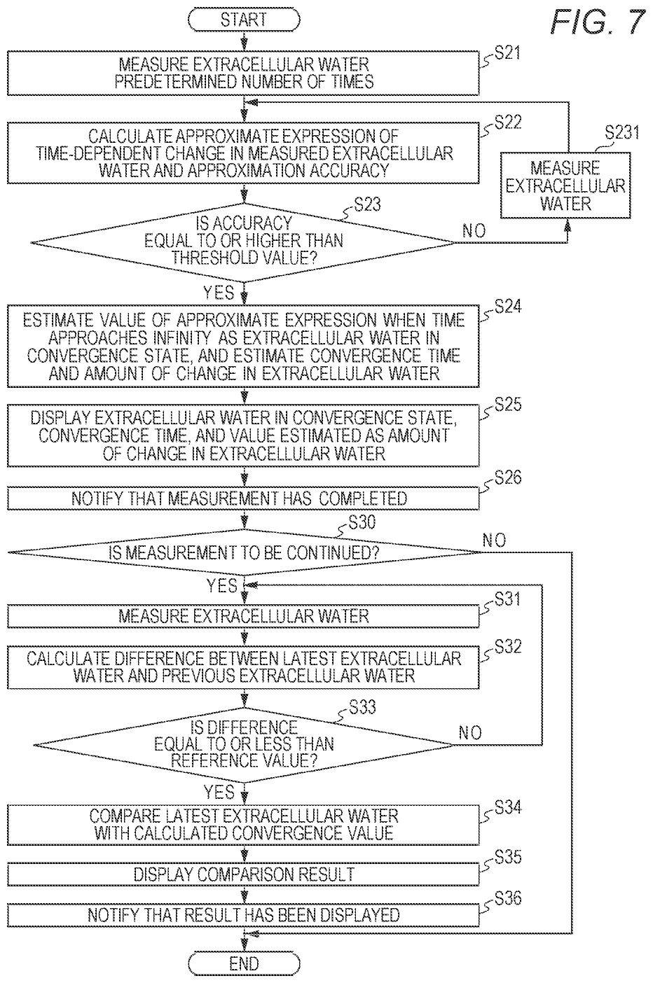

FIG. 7 is a flowchart of a measurement method according to a modification example of the second embodiment.

The measurement device 10 and the measurement method according to the modification example are different from the measurement device 10 and the measurement method according to the second embodiment in that the measurement of the extracellular water can be continued after step S26. Hereinafter, the measurement device 10 and the measurement method according to the modification example will be described. In addition, since the processing up to step S26 is the same as the measurement method according to the second embodiment, the description thereof will be omitted.

After step S26, the control section 220 instructs the measurer D to select whether or not to continue the measurement through the display section 250 (step S30).

When the measurement is not to be continued (S30; No), the control section 220 ends the measurement operation.

When the measurement is to be continued (S30; Yes), the control section 220 causes the measurement section 210 to measure the extracellular water (step S31).

Then, the estimation section 222 calculates the difference ΔECWₙ between the latest (n-th) extracellular water and the previous ((n−1)-th) extracellular water (step S32).

Then, the estimation section 222 determines whether or not the difference ΔECWₙ between the latest (n-th) extracellular water and the previous ((n−1)-th) extracellular water (step S33).

When the difference ΔECWₙ is not equal to or less than the reference value (S33; No), the control section 220 repeatedly performs steps S31 to S33 until the difference ΔECWₙ becomes equal to or less than the reference value.

When it is determined that the difference ΔECWₙ is equal to or less than the reference value (S33; Yes), the analysis section 223 compares the latest (n-th) extracellular water with the convergence value ECW_{∞} (step S34). The analysis section 223 performs a comparison by calculating the difference or ratio between the latest (n-th) extracellular water and the convergence value ECW_{∞}.

Then, the control section 220 causes the display section 250 to display the comparison result between the measurement value of the latest extracellular water and the convergence value ECW_{∞} (step S35). As a result, it is possible to verify the validity of the convergence value ECW_{∞}.

Then, the control section 220 causes the notification section 260 to notify that the comparison result has been displayed (step S36).

In addition, when the approximation accuracy does not become equal to or higher than the threshold value even if a predetermined amount of time (for example, 20 minutes) has elapsed from the start of measurement and/or when the difference ΔECWₙ does not become equal to or less than the reference value even if a predetermined amount of time (for example, 20 minutes) passes from the start of measurement, the estimation section 222 may stop the estimation operation, and the control section 220 may display the measurement value of the latest extracellular water as a reference value on the display section 250 of the control unit 200 or the display section of the operation terminal 20 of the measurer D together with the fact that the estimation operation has stopped.

As described above, the measurement device 10 may continue the measurement after estimating the extracellular water in the convergence state using Approximate Expression F.

While the present invention has been described above with reference to the embodiments and the modification example, the present invention is not limited to the respective configurations described above, and can be appropriately modified based on the description of the claims.

For example, the means and method for performing various kinds of processing in the measurement device may be realized by a dedicated hardware circuit or a programmed computer.

In addition, in the embodiments described above, the form has been described in which the control unit 200 functions as the estimation section 222 and the analysis section 223. However, the operation terminal 20 of the measurer D may function as the estimation section 222 and the analysis section 223. In addition, the measurement section 210, the estimation section 222, and the analysis section 223 of the measurement device 10 have been described as being implemented in one device (control unit 200), but the device configuration is not limited thereto. For example, in the measurement device 10, the measurement section 210 may be part of the control unit 200, and the estimation section 222 and the analysis section 223 may be parts of other devices (e.g., operation terminal of the measurer D, one or more servers, and a cloud server).

In addition, subjects of the body water content measurement device, method, and program according to the present invention are not limited to patients with heart failure or renal failure.

In addition, the body water content measured by the body water content measurement device, method, and program according to the present invention is not limited to the extracellular water, and may be the intracellular water or may be the total amount of water that is the sum of the extracellular water and the intracellular water.

What is claimed is:

1. A body water content measurement device, comprising:
a measurement section configured to generate a plurality of measurements corresponding to body water content of a subject over time; and
an estimation section configured to estimate the body water content in a convergence state based on a plurality of body water content calculated respectively from the plurality of measurements.

2. The body water content measurement device according to claim 1, wherein
the estimation section calculates a difference in first and second body water content calculated respectively from first and second measurements generated successively by the measurement section, and
when the difference is equal to or less than a reference value, the estimation section estimates the second body water content as the body water content in the convergence state.

3. The body water content measurement device according to claim 1, wherein the estimation section calculates an approximate expression that is a function of time for approximating the body water content from the plurality of body water content calculated respectively from the plurality of measurements, and estimates, as the body water content in the convergence state, a value of the approximate expression when time approaches infinity.

4. The body water content measurement device according to claim 3, wherein
the estimation section calculates an approximation accuracy of the approximate expression, and
when the approximation accuracy is equal to or higher than a threshold value, the estimation section sets, as the body water content in the convergence state, the value of the approximate expression when time approaches infinity.

5. The body water content measurement device according to claim 1, further comprising:
a time analysis section configured to estimate a convergence time as time elapsed from start of measurement by the measurement section to when the body water content in the convergence state is reached.

6. The body water content measurement device according to claim 1, further comprising:
a body water content analysis section configured to estimate an amount of change in the body water content during a time period that extends from start of measurement by the measurement section to when the body water content in the convergence state is reached.

7. The body water content measurement device according to claim 1, further comprising:
a notification section configured to notify that the estimation of the body water content in the convergence state by the estimation section has completed.

8. The body water content measurement device according to claim 1, wherein the measurement section is configured to supply a current to a pair of current application electrodes attached to the body of the subject and measure a voltage difference between a pair of measurement electrodes attached to the body of the subject.

9. The body water content measurement device according to claim 8, wherein a bioimpedance of the subject is calculated from a value of the current supplied to the pair of current application electrodes and the measured voltage difference between the pair of measurement electrodes.

10. The body water content measurement device according to claim 1, wherein the body water content is extracellular water.

11. The body water content measurement device according to claim 10, wherein the extracellular water is calculated from the bioimpedance of the subject, and the height, weight, sex, and age of the subject.

12. A body water content measurement method, comprising:
generating a plurality of measurement corresponding to body water content of a subject over time; and
estimating the body water content in a convergence state based on a plurality of body water content calculated respectively from the plurality of measurements.

13. A non-transitory computer readable medium in which a body water content measurement program is stored, wherein the body water content measurement program is executable on a processor to carry out:
a procedure for acquiring a plurality of measurements corresponding to body water content of a subject over time; and
a procedure for estimating the body water content in a convergence state based on a plurality of body water content calculated respectively from the plurality of measurements.

14. A device for measuring body water content of a subject, comprising:
a pair of first electrodes to be attached to the subject;
a pair of second electrodes to be attached to the subject;
a measurement circuit configured to supply a current to the first electrodes and to measure a voltage difference between the second electrodes; and
a processor configured to calculate a bioimpedance at a plurality of different timings, wherein the bioimpedance is calculated at each timing from a value of the current supplied to the first electrodes at said timing and the voltage difference measured at said timing, and determine the body water content of the subject from a first bioimpedance and a second bioimpedance that are calculated at successive timings.

15. The device according to claim 14, wherein the processor determines extracellular water as the body water content and the extracellular water is calculated from the bioimpedance of the subject, and the height, weight, sex, and age of the subject.

16. The device according to claim 14, wherein the processor calculates a first extracellular water from the first bioimpedance and a second extracellular water from the second bioimpedance, and determines the second extracellular water as the body water content when a difference between the second extracellular water and the first extracellular water is less than or equal to a reference value.

17. The device according to claim 16, wherein the processor is configured to issue a notification upon determining the second extracellular water as the body water content.

18. The device according to claim 16, further comprising:
an interface circuit for wirelessly communicating the body water content to an external device.

19. The device according to claim 14, wherein the processor calculates an approximate expression that is a function of time for approximating the body water content of the subject, based on a plurality of bioimpedances calculated at the different timings, and determines a value of the approximate expression when time approaches infinity as the body water content of the subject.

20. The device according to claim 19, wherein the processor calculates an approximation accuracy of the approximate expression, and sets the value of the approximate expression when time approaches infinity as the body water content of the subject when the approximation accuracy is equal to or higher than a threshold value.
